(19) United States
(12) Patent Application Publication
(10) Pub. No.: US 2025/0152004 A1
(43) Pub. Date: May 15, 2025

(54) METHOD, SYSTEM AND STORAGE MEDIUM FOR EVALUATING EYE MOTION

(71) Applicant: SHANGHAI BAIYI HEALTHCARE TECHNOLOGY CO., LTD ., Shanghai (CN)

(72) Inventors: Chaonan TIAN, Shanghai (CN); Youxiang WANG, Shanghai (CN); Youzhi WANG, Shanghai (CN); Dong DU, Shanghai (CN)

(21) Appl. No.: 19/019,491

(22) Filed: Jan. 14, 2025

Related U.S. Application Data
(63) Continuation of application No. PCT/CN2023/107803, filed on Jul. 18, 2023.

(30) Foreign Application Priority Data
Aug. 18, 2022 (CN) 202210989508.3

(51) Int. Cl. A61B 3/113; A61B 3/00; A61B 3/11

(52) U.S. Cl. CPC A61B 3/113; A61B 3/0025; A61B 3/112

(57) ABSTRACT

A method, system and storage medium for evaluating the angle of ocular movement are disclosed. The evaluation method includes capturing a first gaze bitmap of the user looking straight ahead at a position in front of the eyes in a near-infrared light band of 700-1200 nm, capturing a second eye gaze bitmap and a third eye gaze bitmap of the user's eye moving to a limit position in the direction(s) to be measured, comparing the first gaze bitmap respectively with the second eye gaze bitmap and the third eye gaze bitmap, and calculating the angle of ocular movement. The eye images are taken in near-infrared light, which effectively differentiates the iris from the pupil so as to accurately identify the pupil edge, and then calculates the angle of ocular movement by comparing different eye gaze bitmaps, and further improves the calculation accuracy of the angle of ocular movement through compensation.

### CROSS-REFERENCE TO RELATED APPLICATIONS

This application is a continuation of International Pat. Appl. No. PCT/CN2023/107803, filed on Jul. 18, 2023, which claims priority to Chinese Pat. Appl. No. 202210989508.3, filed on Aug. 18, 2022, the contents of each of which are incorporated by reference herein in their entireties.

### TECHNICAL FIELD

This application belongs to the field of ocular detection technology, and specifically relates to a method, device and storage medium for evaluating eye motion (ocular movement).

### BACKGROUND DISCUSSION

The oculomotor, trochlear and abducens nerves have the function of controlling the movement of the extraocular muscles of the eye. These nerves are part of the motor cranial nerves. When any of the above nerves or nerve nuclei are damaged, alone or together, it can appear as irregular eye movement or diplopia. Complete damage occurs when the external eye muscles are paralyzed, and the eye is fixed. Extraocular muscle injury, or infection or myopathy caused by extraocular muscle paralysis, can also appear as irregular eye motion. Clinically, such conditions are collectively referred to as eye motion disorders. Eye motion disorders may also be associated with medical diseases, including orbital diseases, diabetes, neuroinflammation, etc. Therefore, accurate evaluations of the motion capabilities of both eyes is of great significance for detection of eye disorders.

In the prior art, the detection of eye motion is usually carried out manually by the doctor. Specific detection methods include: 1) Eye motion examination in one eye: cover the opposite eye, start from the first gaze, ask the patient to look at the flashlight, and move along the diagnostic direction of gaze; 2) Eye motion examination of both eyes: starting from the first gaze position, ask the patient to look at the flashlight and move along the diagnostic direction of gaze. Binocular motion examination can evaluate the relative position of the eyes and obtain information different from monocular motion examination. However, in the above detection methods, there is no objective data, and the changes of patients over time are generally not recorded and do not include continuous data.

In addition, the prior art also discloses the use of automatic detection methods to detect eye motion. For example, in Chinese patent application number 202011260253.4, entitled “An eye motion detector” (publication date: Feb. 19, 2021), the head of the patient is fixed using a head fixer, an indicator light in front of the head fixer directs a visible light beam away from the patient's eyes, and the visible light beam extends beyond the patient's static field of vision. An imaging lens in front of the head fixer captures an image of the patient's eye moving along the direction of the visible beam, and the patient's eye movement is obtained from the image of the eye captured by the imaging lens. However, in the above scheme, the eye image is not specifically distinguished and processed, resulting in insufficient detection accuracy of eye movement. In another example, in Chinese patent application no. 201710054692.1, entitled “A computer-based device and method for detecting eye motion distance and binocular motion consistency deviation” (publication date: Jun. 13, 2017), a limbal extraction unit extracts baseline photos from the camera and the limbus in each test photo, and calculates the movement distance of the eyeball in all directions through extracted points on the limbus, so as to calculate a deviation in binocular motion or binocular motion consistency. However, because the limbus is the transitional zone between the cornea and the sclera, the extracted boundary of the limbus is not sufficiently clear, which affects the accuracy of the final result.

### SUMMARY

Purposes of this application include providing a method and system for evaluating eye motion, aiming at improving the accuracy of eye motion evaluations and reducing or eliminating errors in the measurement process. By collecting multiple different eye gaze bitmaps of a user or patient in a near-infrared light band, and then analyzing the collected images of multiple different eye positions, angles of ocular movement are calculated, and accurate evaluations of eye motion are achieved.

In order to achieve the above purposes, on the one hand, this application provides a method of evaluating eye motion, which includes the following steps:
in a near-infrared light band of 700-1200 nm, capturing a first gaze bitmap of an eye of a user (e.g., a patient) looking straight ahead at a position in front of the user's eyes (Step S1);
capturing a second eye gaze bitmap and a third eye gaze bitmap of the user's eye moving to a limit position in one or more directions to be measured (Step S2); and
respectively comparing the first gaze bitmap with the second eye gaze bitmap and the third eye gaze bitmap to calculate an angle of ocular movement (Step S3).

Furthermore, the directions to be measured may include eyes moving upward, downward, inward, outward, inward up, outward up, inward down and outward down.

Furthermore, respectively comparing the first gaze bitmap with the second eye gaze bitmap and the third eye gaze bitmap may comprise adopting a convolutional neural network to segment the pupil from the images of the first gaze bitmap, the second eye gaze bitmap and the third eye gaze bitmap.

Respectively comparing the first gaze bitmap with the second and third eye gaze bitmaps may further comprise taking a center C₁ of the first eye gaze bitmap and a center C₂ of either the second eye gaze bitmap or the third eye gaze bitmap, and connecting the center C₁ and the center C₂ with a first straight line;

finding edge points P₁ and P₂ at a same point on an edge of the pupil in the first eye gaze bitmap and either the second eye gaze bitmap or the third eye gaze bitmap (e.g., whichever bitmap contains the center C₂) along the first straight line, where the edge points P₁ and P₂ are preferably along the first straight line;

connecting the edge points P₁ and P₂ to form a second straight line (or line segment) P₁P₂;

dividing the second straight line (or line segment) P₁P₂ into a third line (or line segment) A and a fourth line (or line segment) B by a fifth line perpendicular to the second line P₁P₂ and passing through the center C₁; and

using the radius of the eye r, obtaining a first angle α corresponding to the third line (or line segment) A and a second angle β corresponding to the fourth line (or line segment) B, and obtaining an angle of ocular movement by adding the first angle α and the second angle β.

Furthermore, the angle of ocular movement may be compensated by an amplitude of pupil change, an amplitude of the center shift of the eye, and/or a corneal refraction error compensation in the first gaze bitmap, the second eye gaze bitmap or the third eye gaze bitmap.

Furthermore, the angle of ocular movement may be compensated by the amplitude of pupil change by adjusting a position of the edge point P₂ by an amount corresponding to the amplitude of pupil change in the second gaze bitmap or the third gaze bitmap (whichever bitmap is selected for compensation) relative to the first gaze bitmap.

In a more specific embodiment, the pupil change is a change in the diameter of the pupil, calculated as a difference between the diameter of the pupil in the first gaze bitmap and the diameter of the pupil in the second eye gaze bitmap or the third eye gaze bitmap (whichever bitmap is selected for compensation) along the direction to be measured, and the amount by which the position of the edge point P₂ is compensated corresponds linearly to the amplitude of the pupil change.

Furthermore, the angle of ocular movement may be compensated by the amplitude of the center shift of the eye (e.g., the displacement of the center of the eye), and more specifically by obtaining an overlapping image of the eye in the first gaze bitmap and at least one of the second eye gaze bitmap and the third eye gaze bitmap, determining the amplitude of the center shift of the eye from the overlapping image, and adjusting the angle of ocular movement by an amount related to the amplitude of the center shift of the eye in the direction to be measured.

Furthermore, the angle of ocular movement may be compensated by a corneal refraction error compensation, and more specifically by compensating the angle of ocular movement linearly with the angle of ocular movement. In other words, the corneal refraction error compensation is linearly related to the angle of ocular movement (i.e., CREC=c*ϕ, where CREC is the corneal refraction error compensation, c is a constant, and ϕ is the uncompensated angle of ocular movement).

In a further aspect, the application discloses a system for evaluating eye motion, comprising:
a first acquisition module, configured to capture a first gaze bitmap of an eye of a user looking straight ahead at a position in front of the user's eyes in a near-infrared light band of 700-1200 nm;
a second acquisition module, configured to capture a second eye gaze bitmap and a third eye gaze bitmap of the user's eye moving to a limit position in one or more directions to be measured, where the first acquisition module and the second acquisition module may both include the same camera; and
an image processing module, configured to compare the first gaze bitmap with the second eye gaze bitmap and the third eye gaze bitmap respectively to calculate an angle of ocular movement. The image processing module may be configured to compensate the angle of ocular movement using one or more of the compensation techniques described herein.

In another aspect, the application discloses a program code stored in a computer-readable storage medium, which is loaded and/or executed by a processor to implement the present method of evaluating eye motion. The processor that loads and/or executes the program code (e.g., a set of computer-readable and/or processor-executable instructions) and/or the computer-readable storage medium may be included in the image processing module.

Compared with the prior art, the disclosed technical solution(s) have at least the following beneficial effects:

The iris and pupil can be effectively distinguished by taking eye images (e.g., at a wavelength or subband) in a near-infrared light band, so as to accurately identify the edge of pupil. By using the displacement of pupil edge points to calculate the angle of ocular movement, the shortcomings of using an iris edge to calculate the angle of ocular movement in the prior art are overcome.

Further, in this application, the amplitude of pupil change before and after eye motion, the amplitude of eye center shift or displacement, and/or a corneal refraction error compensation are used to compensate the angle of ocular movement, which can further improve the accuracy of calculations of the angle of ocular movement, so as to accurately calculate the angle of ocular movement in each direction to be measured, and provide a more accurate basis for detecting and/or determining an extent of eye disorders.

### DESCRIPTION OF THE DRAWINGS

In order to more clearly illustrate the technical scheme of the embodiment(s) of this application, a brief introduction of the drawings to be used in the description of the embodiment(s) of this application or the prior art is disclosed below.

FIG. 1 is a flow chart of an exemplary method for evaluating eye motion according to this application;

FIGS. 2A-B show images of an eye taken in visible light (FIG. 2A) and near-infrared light (FIG. 2B);

FIGS. 3A-I show overlay images of the second or third eye gaze bitmap with the first eye gaze bitmap, taken in each of eight (8) different directions to be measured;

FIG. 4 shows an exemplary calculation of the angle of ocular movement;

FIG. 5 shows the pupillary changes before and after eye movement;

FIG. 6 shows the overlapping images of eye imaging before and after eye motion;

FIGS. 7A-B show the displacement of the center of the eye before and after eye motion;

FIG. 8 shows the refraction of the corneal lens on the pupil;

FIGS. 9A-B show the angle of ocular movement calculated using the edge of the pupil (FIG. 9B) and the edge of the iris (FIG. 9A);

FIG. 10 shows a structure diagram of an exemplary device for evaluating eye motion.

### EXAMPLES

Below, combining the attached drawings and embodiments, a further detailed description of one or more specific implementations of this application are provided. The following embodiments are used to illustrate, but not to limit, the scope of the application.

### Example 1

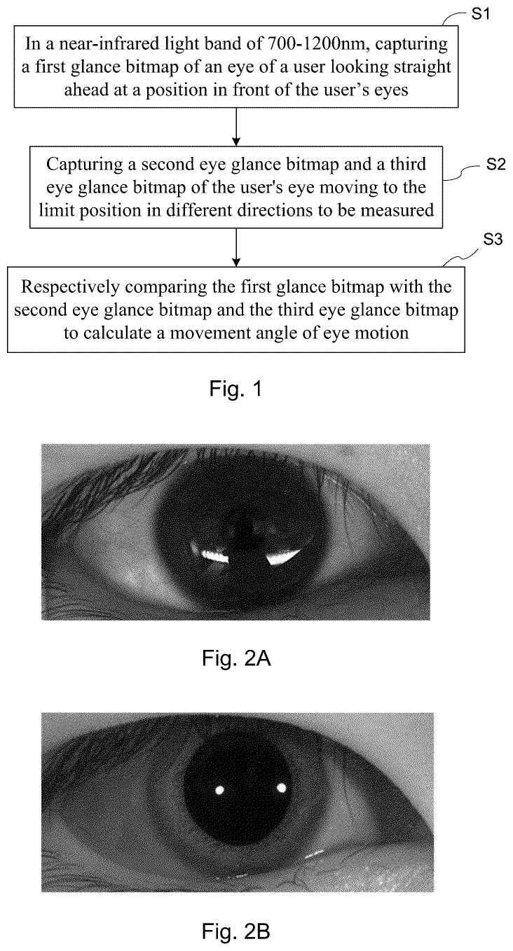

On the one hand, as shown in FIG. 1, this embodiment 1 provides a method of evaluating for eye motion that includes:

S1: in a near-infrared light band of 700-1200 nm, capturing a first gaze bitmap of a user looking straight ahead at a position in front of the eyes;

S2: capturing a second eye gaze bitmap and a third eye gaze bitmap of the user's eye moving to the limit position in a direction to be measured; and

S3: comparing the first gaze bitmap respectively with the second eye gaze bitmap and the third eye gaze bitmap to calculate an angle of ocular movement.

In the method, when an acquisition device is used to obtain the eye image, the user's head may be fixed first in a forehead and/or jaw support, using fixation points in the support and/or at the corner of the eye to achieve fixation of the user's head. After fixing, the first gaze bitmap of the user's eyes facing forward is taken in an infrared red-light band of 700-1200 nm, and the second eye gaze bitmap and the third eye gaze bitmap of the user's eyes moving to the limit position in the direction indicated by an indicator light (e.g., in the acquisition device, or fixed or connected to the forehead and/or jaw support) in each direction to be measured are taken.

It should be noted that the eye position refers to the position of the eyeball during the eye examination, which includes the first gaze position, the second eye position, and the third eye position. The first eye position refers to the eye position when the two eyes look straight ahead towards an infinite distance in a horizontal plane, the second eye position refers to the eye position when the eyeball moves up, down, in and out, and the third eye position refers to the eye position when the eyeball moves inwardly up, inwardly down, outwardly up and outwardly down, that is, the eye position when the eyeball moves up, under the nose, supratemporal and sub-temporal. Accordingly, the first, second and third eye gaze bitmaps refer to the images taken by the eyeball at each eye position, respectively.

As shown in FIG. 2A, in the normal visible light band of 400-700 nm, the colors of different parts of the eye, namely the pupil, iris, and sclera, may have little effect on imaging. Due to the gradual structure of the corneal limbus in the interface between the iris and sclera, it is difficult or impossible to accurately identify the spherical center of the eye. The peak absorption of human melanin pigment occurs at about 335 nm, and wavelengths over 700 nm are almost completely reflective and/or transparent. Thus, the reflectivity of the iris is quite stable in near-infrared wavelength bands over 700 nm (see, e.g., FIG. 2B). Therefore, the use of near-infrared light can easily distinguish the sclera, iris and pupil boundaries. Thus, the accuracy and stability of the measurement method (e.g., measurement algorithm) can be improved.

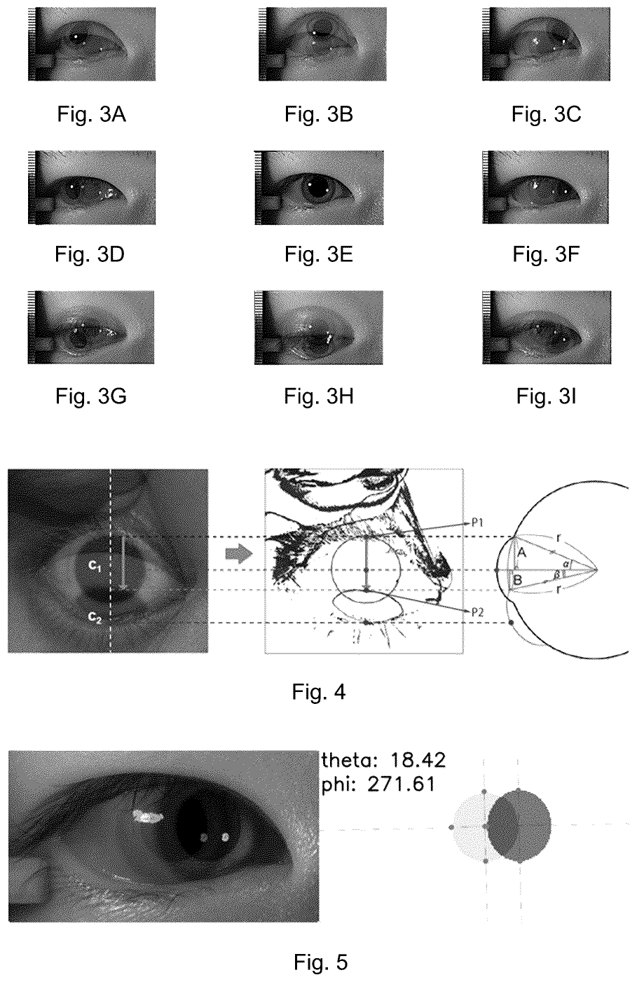

As shown in FIGS. 3A-I, the directions to be measured include eye upward (FIG. 3B), downward (FIG. 3H), inward (FIG. 3F), outward (FIG. 3D), inward up (FIG. 3C), outward up (FIG. 3A), inward down (FIG. 3I) and outward down (FIG. 3G). An image of the eye facing straight ahead (FIG. 3E) is used as a reference, and it appears as a “shadow image” in each of FIGS. 3A-D and 3F-I. Angles of ocular movement of each eye in each of the eight directions can be measured, but at least two directions (preferably opposite directions, such as up and down, inward and outward, etc.) or four directions (preferably two pairs of opposite directions) are measured. Each time one eye is measured, the other eye is shielded, and the indicator light set in each direction to be measured is used to instruct the eye (i.e., the left eye and then the right eye, or vice versa) to rotate in each direction. The second eye gaze bitmap and the third eye gaze bitmap are obtained and compared with the first eye gaze bitmap as the benchmark (e.g., as shown in FIGS. 3A-D and 3F-I), so as to calculate the angle(s) of ocular movement.

It should be noted that the above angle(s) of ocular movement refer to the maximum angle of rotation of the eye sphere around the center of the eye before and after the eye motion (e.g., after the eye motion, relative to before the eye motion [e.g., as shown in FIG. 3E]).

### Example 2

As shown in FIG. 4, based on embodiment 1 of the above method, this embodiment 2 further defines the calculation method of the angle of ocular movement, specifically:
adopting a convolutional neural network to segment the pupil from the images of the first gaze bitmap, the second eye gaze bitmap and the third eye gaze bitmap;
taking a center C₁ of the first eye gaze bitmap and a center C₂ of the second eye gaze bitmap or the third eye gaze bitmap, and connecting the center C₁ and the center C₂ with a first straight line;
finding edge points P₁ and P₂ (e.g., along the edge of the pupil) before and after movement of the pupils in the first eye gaze bitmap and either the second eye gaze bitmap or the third eye gaze bitmap (whichever contains the center C₂) along the first straight line;
connecting the edge points P₁ and P₂ to form a second straight line or line segment P₁P₂;
dividing the second straight line or line segment P₁P₂ into two segments, namely a third line or line segment A and a fourth line or line segment B, by a fifth line perpendicular to the second straight line P₁P₂ and passing through the center C₁;
using the radius of the eye r, obtaining a central angle α corresponding to the third line or line segment A and a central angle β corresponding to the fourth line or line segment B, and obtaining the angle of ocular movement by adding the central angle α and the central angle β.

In the above calculation method, the angle of ocular movement before and after the eye movement can be obtained by calculating the central angle(s) corresponding to the arc length of the sphere through simple analytic geometric relations. It should be noted that since the eyeball is a regular or substantially regular spherical structure, in each direction to be measured, such as the inside up, inside down, outside up, and outside down directions of the oblique movement of the eyeball, the angle of ocular movement can be calculated by image and data processing steps after obtaining the first, second, and third eye gaze bitmaps.

### Example 3

As shown in FIGS. 5-7, based on the above method in embodiment 2, this embodiment 3 further defines methods of compensating the angle of ocular movement, thereby improving the accuracy of calculating the angle(s) of ocular movement. In the actual measurement, it is found that changes in the pupil (or its shape) before and after eye movement, a displacement of the center of the eye (e.g., as a result of contraction of the muscles moving the eye), and the influence of corneal refraction may cause errors in the calculation of the angle(s) of ocular movement. The compensation methods for the above errors are explained in detail below.

First of all, as shown in FIG. 5, when the eye rotation deviates from orthotropia, the image of the pupil may become elliptical, and the flattening direction will be in the direction of rotation, while an image perpendicular to the direction of rotation (e.g., perpendicular to a plane passing through the edge of the pupil) will not be affected. So, if there is a change in the diameter of the pupil as the eye moves in the direction of rotation, it comes from the change of the pupil shape. If the orthotropia is smaller than the strabismus, the pupil is dilated when it rotates, and if the orthotropia is larger than the strabismus, the pupil shrinks when it rotates. For example, if the ellipse perpendicular to the direction of rotation is shrinking, it comes from the contraction of the pupil. As shown in the right-hand side of FIG. 5, the two centers of the separated circles/ellipses (i.e., the pupils before and after movement to the limit position) are connected in a straight line, the diameters of the two circles/ellipses along and/or perpendicular to the straight line are measured, the proportion of the pupil contraction is calculated using the diameters (e.g., along the straight line, and optionally perpendicular to the straight line to calculate a reduction in area), and at least one of the edge points of the circle and/or ellipse is corrected (e.g., by an amount equal to the proportional reduction in diameter along the line or in the area of the circle/ellipse). For example, the position of the edge point P₂ can be compensated by the amplitude of the pupil change (e.g., the reduction in diameter along the line), thereby eliminating the influence of pupil contraction on the accuracy of calculating the angle of ocular movement, so that the amplitude of the pupil change in the second eye gaze bitmap or the third eye gaze bitmap relative to the first gaze bitmap can compensate the angle of ocular movement.

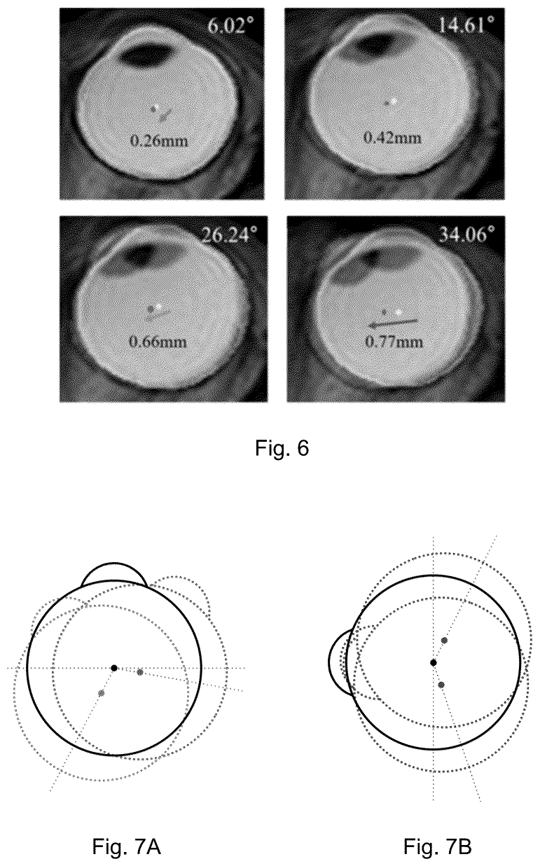

As shown in FIG. 6, when the eyes are rotating, the eyes are not suspended in space. Due to the soft tissues around them and the uneven power of the eye muscles that move the eye, eye rotation in different directions causes different degrees of displacement of the eye center. The greater the angle of eye rotation, the more the center of the eye shifts. The effect of the displacement of the center of the eye on the algorithm is equivalent to the misalignment of the image when overlapping the front and side views (e.g., superimposing the image of the eye in the limit position along the direction to be measured and the image of the eye looking straight ahead), resulting in the calculated angle of ocular movement being too large or too small. This method of compensation determines how much the center of the eye shifts using overlapping imaging, and reverses the displacement to offset the effect of the eye center shift.

FIG. 7A shows the changes before and after the eye rotates in the inward and outward directions, taken from the direction of the human cranial top, while FIG. 7B shows the changes before and after the eye rotates in the upward and downward directions, taken from the direction of the human cranial top. As the eye rotates (e.g., in the measurement direction), the displacement of the center of the eye towards the front or back (e.g., along a depth direction relative to the imaging lens) has no effect on the image taken by the camera in front of it, so it can be ignored. Only images projected by the eye in the cross-section (e.g., a plane defined by up-down and left-right axes relative to the imaging lens) are taken. Statistically, the relationship between the direction of the eye movement and the displacement of the center of the eye in the cross-section is as follows:
30° inward rotation of the eyeball: 0.69 mm inward displacement of the eyeball center
30° outward rotation of the eyeball: 0.45 mm outward displacement of the eye center;
20° up rotation of the eyeball: 0.43 mm downward displacement of the eye center;
20° down rotation of the eyeball: 0.43 mm upward displacement of the eye center.

In addition, the displacement of the eye center and the rotation angle of the eye are linear or substantially linear changes. If the rotation angle of the eye is 0° (straight version), the displacement of the eye center is 0 mm. The position of the center of the eyeball C₂ in the calculation of the angle of ocular movement can be corrected using the relationship between the eye center displacement or shift and the rotation angle calculated in advance (i.e., without compensation) so as to compensate the angle of ocular movement by the magnitude of the eye center displacement in the second eye gaze bitmap or the third eye gaze bitmap relative to the first gaze bitmap.

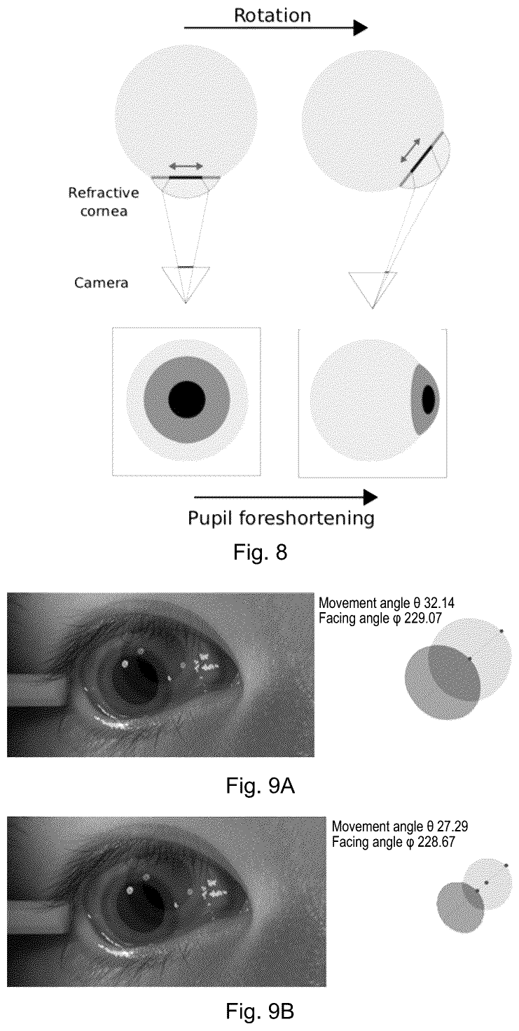

As shown in FIG. 8, when calculating the angle of ocular movement, the edge point of the pupil can be affected by the refraction of the corneal lens, and the edge point of the pupil in the picture is not the real position (e.g., as a result of this refraction). On the other hand, the outer edge point of the iris is not affected, because it is not covered by the cornea. In the same photo (shown in both FIGS. 9A and 9B), two calculations based on the pupil edge point (FIG. 9B) and the iris edge point (FIG. 9A) result in different angles of ocular movement, but the calculation using the pupil edge point (FIG. 9B) has an error due to corneal refraction. Through experimental measurement, it has been found that the difference between the angles of ocular movement calculated using the pupil edge point and using the iris edge point is substantially linear, so the formula to compensate for the corneal refraction error in the calculation of the angle of ocular movement is as follows:

Compensation of angle of ocular movement θ=0.13134×angle of ocular movement θ+0.52704

Corrected angle of ocular movement θ=angle of ocular movement θ+compensation of angle of ocular movement θ

In the above formula, the angle of ocular movement θ is the calculated angle of ocular movement before corneal refraction error compensation.

After taking into account the changes of the pupil, the displacement of the center of the eye, and the influence of corneal refraction on the angle of ocular movement before and after the above eye movement, errors in the calculation of the angle of ocular movement can be eliminated and an accurate angle of ocular movement can be obtained.

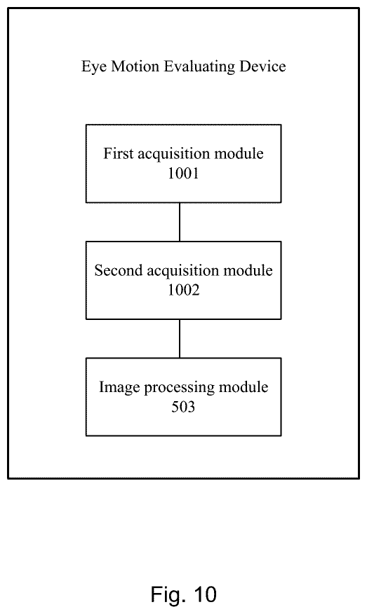

In another aspect, this application discloses a device for evaluating eye motion, as shown in FIG. 10, including: a first acquisition module 1001, a second acquisition module 1002 and an image processing module (1003), in which:

The first acquisition module 1001 is configured to capture a first gaze bitmap of a user looking straight ahead in front of the eyes in a near-infrared light band of 700-1200 nm;

The second acquisition module 1002 is configured to capture a second eye gaze bitmap and a third eye gaze bitmap of the user's eyes moving to a limit position in a measured direction in front of the eyes; and

The image processing module 1003 is configured to compare the first gaze bitmap with the second eye gaze bitmap and the third eye gaze bitmap respectively to calculate an angle of ocular movement. The image processing module may be configured to compensate the angle of ocular movement using one or more of the compensation techniques described herein. In addition, the first acquisition module and the second acquisition module may both include the same camera.

On the one hand, the present application discloses a computer-readable storage medium in which at least one program code is stored, which is loaded and executed by a processor to implement the operations performed in the method of evaluating eye motion.

With regard to the device for evaluating eye motion in the above embodiments, the specific ways in which each module performs operations have been described in detail in the method embodiments. Please refer to the part of the method embodiments for relevant description.

In an exemplary embodiment, a computer-readable storage medium is also disclosed, including a memory storing at least one program code that is loaded and executed by the processor to perform the method of evaluating eye motion in the embodiments. For example, the computer-readable storage medium can be Read-Only Memory (ROM), Random Access Memory (RAM), Compact Disc Read-Only Memory (CDROM), magnetic tape, floppy disk and optical data storage devices, etc.

A person of ordinary skill in the art may understand that all or part of the steps to implement the above embodiments may be performed by hardware, or by a program in hardware related to at least one program code, which may be stored in a computer-readable storage medium, such as read-only memory, disk or optical disc, etc.

The above examples are only preferred embodiments of this application and are not intended to limit this application. Any modification, equivalent replacement, improvement, etc. made within the spirit and principles of this application shall be covered by the protection scope of this application.

What is claimed is:

1. A method of evaluating eye motion, comprising:
in a near-infrared light band of 700-1200 nm, capturing a first gaze bitmap of an eye of a user looking straight ahead at a position in front of the user's eyes;
capturing a second eye gaze bitmap and a third eye gaze bitmap of the eye of the user moving to a limit position a direction to be measured; and
respectively comparing the first gaze bitmap with each of the second eye gaze bitmap and the third eye gaze bitmap to calculate an angle of ocular movement.

2. The method of evaluating eye motion of claim 1, wherein the direction to be measured includes a plurality of directions.

3. The method of evaluating eye motion of claim 2, wherein the plurality of directions include upward, downward, inward, outward, inward up, outward up, inward down and outward down.

4. The method of evaluating eye motion of claim 1, wherein comparing the first gaze bitmap with each of the second eye gaze bitmap and the third eye gaze bitmap comprises:
segmenting the pupil in the images of the first gaze bitmap, the second eye gaze bitmap and the third eye gaze bitmap;
taking a center C₁ of the pupil in the first eye gaze bitmap and a center C₂ of the pupil in either the second eye gaze bitmap or the third eye gaze bitmap, and connecting the center C₁ and the center C₂ with a first straight line;
finding edge points P₁ and P₂ at a same point on an edge of the pupil in the first eye gaze bitmap and either the second eye gaze bitmap or the third eye gaze bitmap along the first straight line;
connecting the edge points P₁ and P₂ to form a second straight line P₁P₂;
dividing the second line P₁P₂ into a third line A and a fourth line B by a fifth line perpendicular to the second straight line P₁P₂ and passing through the center C₁; and
using a radius of the eye r, obtaining a first angle α corresponding to the third line A and a second angle β corresponding to the fourth line B, and obtaining the angle of ocular movement by adding the first angle α and the second angle β.

5. The method of evaluating eye motion of claim 4, further comprising compensating the angle of ocular movement with an amplitude of pupil change, an amplitude of a center shift of the eye, and/or a corneal refraction error compensation.

6. The method of evaluating eye motion of claim 5, wherein the angle of ocular movement is compensated by the amplitude of pupil change, and compensating the angle of ocular movement comprises adjusting a position of the edge point P₂ by an amount corresponding to the amplitude of pupil change in the second gaze bitmap or the third gaze bitmap relative to the first gaze bitmap.

7. The method of evaluating eye motion of claim 6, wherein the pupil change is a change in a diameter of the pupil, calculated as a difference between the diameter of the pupil in the first gaze bitmap and the diameter of the pupil in the second eye gaze bitmap or the third eye gaze bitmap along the direction to be measured, and the amount by which the position of the edge point P₂ is compensated corresponds linearly to the amplitude of pupil change.

8. The method of evaluating eye motion of claim 5, wherein the angle of ocular movement is compensated by the amplitude of the center shift of the eye, and compensating the angle of ocular movement comprises obtaining an overlapping image of the eye in the first gaze bitmap and at least one of the second eye gaze bitmap and the third eye gaze bitmap, determining the amplitude of the center shift of the eye from the overlapping image, and adjusting the angle of ocular movement by an amount related to the amplitude of the center shift of the eye in the direction to be measured.

9. The method of evaluating eye motion of claim 5, wherein the angle of ocular movement is compensated by the corneal refraction error compensation, and the corneal refraction error compensation is linear with the angle of ocular movement.

10. The method of evaluating eye motion of claim 5, comprising compensating the angle of ocular movement with each of the amplitude of pupil change, the amplitude of the center shift of the eye, and the corneal refraction error compensation.

11. The method of evaluating eye motion of claim 1, wherein the second eye gaze bitmap is captured when the eye of the user moves in a first direction, and the third eye gaze bitmap is captured when the eye of the user moves in a second direction different from the first direction.

12. The method of evaluating eye motion of claim 11, wherein the first direction is selected from upward, downward, inward, outward, inward up, outward up, inward down and outward down, and the second direction is opposite from the first direction.

13. A system for evaluating eye motion, it comprises:
a first acquisition module, configured to capture a first gaze bitmap of an eye of a user looking straight ahead at a position in front of the user's eyes in a near-infrared light band of 700-1200 nm;
a second acquisition module, configured to capture a second eye gaze bitmap and a third eye gaze bitmap of the eye of the user moving to a limit position in a direction to be measured; and
an image processing module, configured to respectively compare the first gaze bitmap with the second eye gaze bitmap and the third eye gaze bitmap to calculate an angle of ocular movement.

14. The system for evaluating eye motion of claim 13, wherein the first acquisition module and the second acquisition module comprise a same camera.

15. A non-transitory computer-readable storage medium, comprising at least one program code stored therein that is loaded and executed by a processor to implement the method of evaluating of eye motion as described in claim 1.
